(19) United States
(12) Patent Application Publication
(10) Pub. No.: US 2008/0152675 A1
(43) Pub. Date: Jun. 26, 2008

(54) Dispersion of polyamino acids in a continuous lipid phase

(75) Inventor: Gauthier POULIQUEN, Lyon (FR)

Correspondence Address: PATTON BOGGS LLP, 8484 WESTPARK DRIVE, SUITE 900, MCLEAN, VA, 22102, US

(73) Assignee: Flamel Technologies, S.A., Venissieux Cedex (FR)

(21) Appl. No.: 12/003,095

(22) Filed: Dec. 20, 2007

Related U.S. Application Data
(60) Provisional application No. 60/875,811, filed on Dec. 20, 2006.

(30) Foreign Application Priority Data
Dec. 20, 2006 (FR) 06 11186
Dec. 20, 2007 (EP) PCT/EP2007/064352

(51) Int. Cl. A61K 47/32; A61K 47/36; A61K 9/107

(52) U.S. Cl. 424/400; 514/772.6; 514/777

(57) ABSTRACT

The invention relates to injectable pharmaceutical compositions for the prolonged release of at least one active principle, comprising at least one active principle in an aqueous phase of amphiphilic polymer, said aqueous phase being in the form of a dispersion in a continuous lipid phase. The composition is in the form of a water-in-oil emulsion comprising:
a pharmaceutically acceptable, continuous lipid phase,
an aqueous disperse phase containing at least one amphiphilic polymer and at least one active principle not covalently bonded to said amphiphilic polymer, and
at least one pharmaceutically acceptable surfactant.

### FIELD OF THE INVENTION

The present patent application relates to novel pharmaceutical formulations based on aqueous colloidal suspensions or aqueous dispersions for the prolonged release of one or more active principles, particularly protein and peptide active principles. It further relates to the applications, especially therapeutic applications, of these pharmaceutical formulations. These active pharmaceutical formulations apply to both human and veterinary therapeutics.

### STATE OF THE ART

In the field of the prolonged release of pharmaceutical active principles, especially therapeutic proteins, there is a need in many cases to ensure as far as possible that the patient's plasma concentration of therapeutic protein or therapeutic peptide is close to the value observed in the healthy subject.

This objective is compromised by the short life of proteins in the plasma, which makes it necessary to inject the therapeutic protein repeatedly. The plasma concentration of therapeutic protein then has a “sawtooth” profile characterized by high concentration peaks and very low concentration minima. The concentration peaks, which are very much greater than the basal concentration in the healthy subject, can have very pronounced harmful effects due to the high toxicity of therapeutic proteins such as the interleukin IL2. Furthermore, the concentration minima are below the concentration necessary for a therapeutic effect, so the patient receives poor therapeutic cover and suffers serious long-term side effects.

Also, to ensure that the patient's plasma concentration of therapeutic protein is close to the ideal value for his treatment, the pharmaceutical formulation in question has to allow the prolonged release of the therapeutic protein so as to limit the variations in plasma concentration over time.

Furthermore, this active formulation should preferably satisfy the following specifications already familiar to those skilled in the art:

1—prolonged release of an active and non-denatured therapeutic protein, e.g. human or synthetic protein, so that the plasma concentration of therapeutic protein is maintained at a therapeutic level;

2—sufficiently low injection viscosity to be easily injectable;

3—biocompatible and biodegradable form exhibiting neither toxicity nor immunogenicity and having an excellent local tolerance.

In an attempt to achieve these objectives, one of the best approaches proposed in the prior art was to develop forms for the prolonged release of therapeutic protein(s) that consisted of low-viscosity liquid suspensions of nano-particles loaded with therapeutic protein(s). These suspensions facilitated the administration of native therapeutic proteins.

Thus, Flamel Technologies has proposed a method in which the therapeutic protein is associated with nanoparticles of a copolyamino acid comprising hydro-phobic groups and hydrophilic groups.

U.S. Pat. No. 5,904,936 describes submicronic particles (NPV), with a mean size of between 0.01 and 0.5 μm, and micronic particles (MPV), with a mean size of between 0.5 and 20 μm, of an amphiphilic polyamino acid copolymer comprising at least two types of amino acid, one being neutral and hydrophobic and the other being ionizable. Proteins such as insulin are spontaneously adsorbed onto these particles in aqueous solution. The polyamino acid copolymer is e.g. a block copolymer of poly(L-leucine-block-sodium L-glutamate). Said patent describes the aggregation of NPV into MPV by adding monocationic salts (ammonium sulfate) or polycationic salts (Fe²⁺, Fe³⁺, Zn²⁺, Ca²⁺, AL²⁺, AP³⁺ or Cu²⁺), an acid (HCl) or cationic polymers (polylysine) to a colloidal suspension of poly-Leu/Glu.

Patent application WO-A-2005/033181 discloses linear, amphiphilic, anionic homopolyamino acids which comprise aspartic residues or glutamic residues and whose ends carry hydrophobic groups containing from 8 to 30 carbon atoms. In particular, the hydrophobically modified, telechelic homopolyamino acids are e.g. a poly[GluONa] with PheOC18/C18 ends or a poly[GluONa] with PheOC18/alpha-tocopherol ends. In water, these hydrophobically modified, telechelic homopoly-amino acids spontaneously form a colloidal suspension of nanoparticles which are easily capable of associating, in aqueous suspension at pH 7.4, with at least one active protein (insulin).

The in vivo release time of the active protein(s) (e.g. insulin) vectorized by the suspensions according to U.S. Pat. No. 5,904,936 & WO-A-2005/033181 could profitably be increased.

An increase in release time has been partially achieved by the pharmaceutical forms described in PCT application WO-A-05/051416. In said patent application, a hydrophobically modified colloidal suspension of nanoparticles (0.001-0.5 μm) of poly(sodium L-glutamate) is injected at a concentration such that, after sub-cutaneous injection, a gel forms in situ in the patient on contact with the endogenous albumin. The protein is then released slowly, typically over a period of one week.

If it is necessary to prolong the release time of the protein beyond the period of one week described in the latter patent application, several possibilities may exist.

A first solution consists in increasing the polymer concentration so as to slow down the release of the protein after in vivo injection. Nevertheless, this method is compromised by a sharp increase in the viscosity of the solution that makes it impossible for this system to be injected.

A second solution consists in dispersing the protein in a water-immiscible, injectable lipid phase so as to reduce the diffusion of the protein in the subcutaneous medium. However, this method is compromised by a potential denaturation of the protein on contact with the lipid phase.

Furthermore, patent U.S. Pat. No. 6,235,282 B1 describes an injectable water-in-oil emulsion as an immunogenic adjuvant in vaccine preparations. An active substance from an immunological point of view, or a vaccine antigen, is contained in the aqueous phase of the emulsion. This results in difficulties with the stability of the active substance in the aqueous phase and in risks of denaturation of the active substance at the water-oil interface. Said patent does not relate to the prolonged release of an active principle.

Patent application US 2004/0071716 A1 relates to an adjuvant useful for vaccine formulations. This adjuvant comprises a water-in-oil emulsion. The emulsifier is a polymeric emulsifier or, more precisely, a sequence copolymer of the general formula A-COO-B-OOC-A, in which B is a divalent residue of a water-soluble polyalkylene glycol and A is a residue of a liposoluble complex mono-carboxylic acid. According to the Examples, the viral antigen is present in the aqueous phase, which entails the same problems of stability and risk of denaturation of the viral antigen. Said patent application does not relate to the prolonged release of an active principle.

### BRIEF DESCRIPTION OF THE INVENTION

It is to the inventors' credit to have proposed a low-viscosity lipid form for increasing the release time of a therapeutic protein associated with a polymer, without reducing the stability of the protein and without increasing the viscosity of the therapeutic form beyond an acceptable limit from the point of view of injectability.

The invention relates first and foremost to a pharmaceutical composition for the prolonged release of at least one active principle. The composition comprises at least one active principle in an aqueous phase containing at least one amphiphilic polymer. The aqueous phase is in the form of a dispersion in a continuous lipid phase. More precisely, the composition is in the form of a water-in-oil emulsion comprising:

a pharmaceutically acceptable, continuous lipid phase,

an aqueous disperse phase containing at least one amphiphilic polymer and at least one active principle not covalently bonded to said amphiphilic polymer, and

at least one pharmaceutically acceptable surfactant.

In one variant of the invention, the amphiphilic polymer carries at least one hydrophobic group.

In one variant of the invention, the amphiphilic polymer is an amphiphilic polyamino acid optionally carrying at least one hydrophobic group. Thus, in one variant of the invention, the pharmaceutical composition is in the form of a water-in-oil emulsion comprising the following components:

a pharmaceutically acceptable, continuous lipid phase,

an aqueous disperse phase containing at least one amphiphilic polyamino acid carrying at least one hydrophobic group, and at least one active principle not covalently bonded to said amphiphilic polyamino acid, and

at least one pharmaceutically acceptable surfactant.

In another variant of the invention, the amphiphilic polymer is a poly-saccharide carrying at least one hydrophobic group.

Such a pharmaceutical composition can be administered via the customary routes, especially via at least one of the following routes: oral, nasal, ocular, cutaneous, vaginal, rectal or parenteral. Among the parenteral routes there may be mentioned subcutaneous injection, intramuscular injection, intraperitoneal injection, intradermal injection, intravenous injection, intra-arterial injection, intraspinal injection, intra-articular injection and intrapleural injection.

According to another feature, the invention relates to the use of the different amphiphilic polymers described below, particularly polysaccharides and polyamino acids, in the preparation of a pharmaceutical composition in the form of a water-in-oil emulsion whose aqueous disperse phase contains at least one of these amphiphilic polymers. One variant of the invention consists in using one or more amphiphilic polyamino acids carrying at least one hydrophobic group in the preparation of such a pharmaceutical composition.

### DETAILED DESCRIPTION OF THE INVENTION

### Definitions

“Physical gel in an aqueous medium” is understood as meaning a semisolid state induced by non-covalent physical interactions between molecules, macro-molecules or particles solubilized or dispersed in an aqueous phase. A physical gel can also be defined via viscoelasticity measurements. In this context a physical gel is a system for which the Young's modulus G′ is greater than the loss modulus G″ over a frequency range such that the characteristic relaxation time, defined as the reciprocal of the crossover point of the two moduli, is greater than or equal to 0.1 s and particularly preferably greater than or equal to 10 s.

For the purposes of the invention and throughout the present disclosure, the term “polyamino acid” covers both natural polyamino acids and synthetic polyamino acids comprising more than 10 amino acid residues. In general in the present description, it shall be understood that “the” polyamino acid can refer to a mixture of different polyamino acids used in the pharmaceutical composition.

For the purposes of the invention, the expression “carries” denotes that the carried group, graft or radical in question is a pendant group. In other words, said group is a side group relative to the main chain of the amphiphilic polymer. In the case of polyamino acids consisting of a concatenation of glutamate and/or aspartate residues, the group is a substituent of the carbonyl group in the δ-position of the aspartic residue or in the ε-position of the glutamic residue which carries it. For other amino acid residues, the pendant group is a substituent of the specific side chain of said amino acid residue.

### Amphiphilic Polysaccharide

In one variant of the invention, the amphiphilic polymer can be a modified polysaccharide such as a hydrophobically modified pullulan (cholesterylpullulan, hexadecylpullulan) described in the article by Kuroda et al. (2002) “Hierarchical self-assembly of hydrophobically modified pullulan in water: gelation by networks of nanoparticles”, Langmuir, 18, 3780-3786.

In another variant of the invention, the amphiphilic polysaccharide used is selected from hyaluronans, alginates, chitosans, galacturonans, chondroitin sulfate, dextrans, celluloses and/or their functionalized derivatives. Such polysaccharides are described in the international patent application published under the number WO 2007/034320. In particular, these are hyaluronans, alginates, chitosans, dextrans and/or their derivatives functionalized by at least one imidazolyl radical and at least one hydrophobic group. A description of this type of polysaccharide and the modalities of their synthesis can be found in the international patent application published under the number WO 2007/116143, particularly as regards dextran derivatives.

### Amphiphilic Polyamino Acid

In another variant of the invention, the amphiphilic polymer used is an amphiphilic polyamino acid. In one preferred variant, it is an amphiphilic polyamino acid carrying at least one hydrophobic group.

In one variant of the invention, the polyamino acids used are homopolymers comprising repeat glutamic acid or aspartic acid residues or copolymers comprising a mixture of these two types of residues. These residues can be in salt form, in which case they are glutamate or aspartate residues. The salts formed in this way must be pharmaceutically acceptable. Various examples of counterions that are generally pharmaceutically acceptable are indicated in the remainder of the description. The glutamic acid or aspartic acid residues and their salts can have the D or L configuration. It is also conceivable for a polyamino acid simultaneously to comprise residues having the D configuration and residues having the L configuration. The repeat residues are bonded to one another via their alpha or gamma positions in the case of the glutamate or glutamic residues and via their alpha or beta positions in the case of the aspartic or aspartate residues.

In one variant of the invention, the main polyamino acid chain essentially comprises amino acid residues having the L configuration which are bonded to one another by linkages of the alpha type (i.e. via their alpha positions).

In another variant of the invention, the amphiphilic polyamino acid is formed of monomers derived from aspartic acid (aspartic residues) and/or from glutamic acid (glutamic residues), at least some of these residues carrying grafts containing at least one hydrophobic group [GH]. These polyamino acids are especially of the type described in PCT application WO-A-00/30618.

In one variant of the invention, in addition to grafts containing at least one hydrophobic group [GH], the amphiphilic polyamino acid can carry substituents derived from a histidine residue. In this case, said histidine residue can be bonded to a glutamic or aspartic residue via an amide linkage.

Various types of amphiphilic polyamino acids carrying hydrophobic groups will now be described [GH]. For the sake of simplicity, the general formulae which follow are written in the form of blocks. However, the amphiphilic polyamino acids corresponding to these formulae can be sequence, block or random polymers or copolymers in particular. It is conceivable to combine the different variants, e.g. by choosing an appropriate mixture of the amphiphilic polyamino acids described below or by combining the various types of grafts within one and the same amphiphilic polyamino acid.

Advantageously, the main chain of the polyamino acid is selected from:
an alpha-L-glutamate or alpha-L-glutamic homopolymer;
an alpha-L-aspartate or alpha-L-aspartic homopolymer; and

an alpha-L-aspartate/alpha-L-glutamate or alpha-L-aspartic/alpha-L-glutamic copolymer.

The distribution of the aspartic and/or glutamic units on the main chain of the amphiphilic polyamino acid is such that the resulting polyamino acid is either random or of the block type or of the multiblock type. Similarly, the distribution of the hydrophobic groups on the main chain of the amphiphilic polyamino acid is such that the resulting polyamino acid is either random or of the block type or of the multiblock type. Thus the general formulae (I), (II), (III), (IV) and (V) shown in the remainder of the description must not be interpreted as representing only sequence (or block) copolymers, but also as representing random copolymers or multiblock copolymers.

According to another definition, the amphiphilic polyamino acid used in the composition according to the invention has a molecular weight of between 2000 and 100,000 g/mol and preferably of between 5000 and 40,000 g/mol.

In a first variant, the amphiphilic polyamino acid used in the pharmaceutical composition has general formula (I) below, and its pharmaceutically acceptable salts:

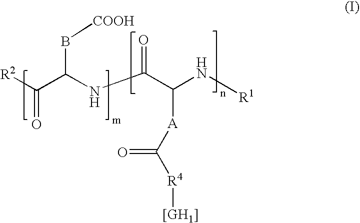

in which:

R¹ is a hydrogen atom, a linear C2 to C10 acyl group, a branched C3 to C10 acyl group, a pyroglutamate group or a group —R⁴-[GH1];

R² is a group —NHR⁵ or a terminal amino acid residue bonded via the nitrogen, whose acid group(s) is (are) optionally modified by an amine —NHR⁵ or an alcohol —OR⁶;

R⁴ independently of one another are a direct bond or a spacer group comprising from 1 to 4 amino acid residues;

R⁵ is a hydrogen atom, a linear C1 to C10 alkyl group, a branched C3 to C10 alkyl group or a benzyl group;

R⁶ is a hydrogen atom, a linear C1 to C10 alkyl group, a branched C3 to C10 alkyl group, a benzyl group or a group —R⁴-[GH1];

A and B independently of one another are a group —CH₂— (aspartic residue) or —CH₂—CH₂— (glutamic residue);

[GH1] is a hydrophobic group;

the molar grafting rate of hydrophobic groups [GH1], n/(n+m), is sufficiently low for the amphiphilic polyamino acid to form a colloidal suspension of submicronic particles of polyamino acid when it is in solution in water at pH 7 and at 25° C., n/(n+m) preferably being between 1 and 25 mol %; and

the degree of polymerization (n+m) varies from 10 to 1000 and preferably from 50 to 300

For further details on the preparation and synthesis of polyamino acids of formula (I), reference may usefully be made to the patent applications published under the numbers FR 2 840 614 and FR 2 855 521.

According to a second possibility, the amphiphilic polyamino acid has one of general formulae (II), (III) and (IV) below, and their pharmaceutically acceptable salts:

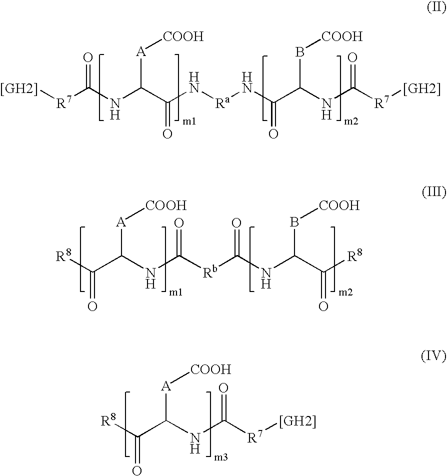

in which:

R^{a} is a linear C2 to C6 alkylene group;

R^{b} is a C2 to C6 alkylene group, a C2 to C6 dialkoxy group or a C2 to C6 diamine group;

R⁷ independently of one another are a direct bond, a spacer group comprising from 1 to 4 amino acid residues, or a group —C(O)—CH₂—CH₂—;

R⁸ are a group —NHR⁹ or a terminal amino acid residue bonded via the nitrogen, whose acid group(s) is (are) optionally modified by an amine —NHR⁹ or an alcohol —OR¹⁰, respectively;

R⁹ is a hydrogen atom, a linear C1 to C10 alkyl group, a branched C3 to C10 alkyl group or a benzyl group;

R¹⁰ is a hydrogen atom, a linear C1 to C10 alkyl group, a branched C3 to C10 alkyl group, a benzyl group or a group —R¹¹-[GH3];

R¹¹ independently of one another are a direct bond or a spacer group comprising from 1 to 4 amino acid residues;

A and B independently of one another are a group —CH₂— (aspartic residue) or —CH₂—CH₂— (glutamic residue);

[GH2] and [GH3] independently of one another are a hydrophobic group; and

the degrees of polymerization (m1+m2) and m3 vary from 10 to 1000 and preferably from 50 to 300.

For further details on the preparation and synthesis of polyamino acids of formulae (II), (III) and (IV), reference may usefully be made to patent application FR2 860 516.

According to a third possibility, the amphiphilic polyamino acid has general formula (V) below, and its pharmaceutically acceptable salts:

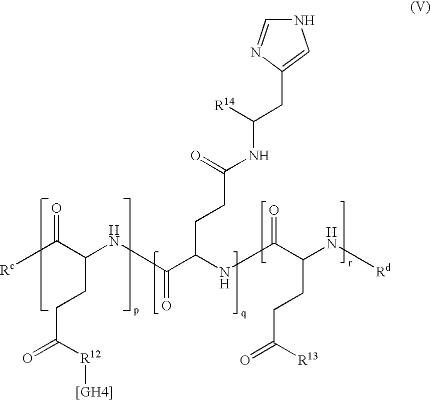

in which:

R^{c} is a group —NHR¹⁵ or a terminal amino acid residue bonded via the nitrogen, whose acid group(s) is (are) optionally modified by an amine —NHR⁵ or an alcohol —OR¹⁶, respectively;

R^{d} is a hydrogen atom, a linear C2 to C10 acyl group, a branched C3 to C10 acyl group or a pyroglutamate group;

R¹² independently of one another are a divalent, trivalent or tetravalent linking group preferably selected from the following groups: —O—, —NH—, C1 to C5-N-alkyl, an amino acid residue, a C2 to C6 diol, a C3 to C6 triol, a C2 to C6 diamine, a C3 to C6 triamine, a C2 to C6 amino alcohol or a C2 to C6 hydroxy acid;

R¹³ independently of one another are a group —OH or an ethanolamine group bonded via the amine fraction;

R¹⁴ is an alkyl ester group, a group —CH₂OH (histidinol), a hydrogen atom (histamine), a group —C(O)NH₂ (histidinamide), a group —C(O)NHCH₃ or a group —C(O)N(CH₃)₂;

R¹⁵ and R¹⁶ independently of one another are a hydrogen atom, a linear C1 to C10 alkyl group, a branched C3 to C10 alkyl group or a benzyl group;

[GH4] independently of one another are each a hydrophobic group selected from:
linear or branched C8 to C30 alkyl groups optionally containing at least one unit of unsaturation and/or at least one heteroatom,
C8 to C30 alkylaryl or arylalkyl groups optionally containing at least one unit of unsaturation and/or at least one heteroatom, and
C8 to C30 (poly)cyclic groups optionally containing at least one unit of unsaturation and/or at least one heteroatom;

p, q and r are positive integers;

the molar grafting rate of hydrophobic groups [GH], (p)/(p+q+r), varies from 1 to 50 mol %, with the proviso that each copolymer chain has an average of at least 3 hydrophobic grafts;

the molar grafting rate of groups derived from the histidine residue, (q)/(p+q+r), varies from 1 to 99 mol %;

(r)/(p+q+r) varies from 0 to 98 mol %; and

(p+q+r) varies from 10 to 1000 and preferably between 30 and 500.

In general, the heteroatoms that can be found in the hydrophobic groups [GH4] are oxygen, nitrogen or sulfur atoms.

The derivatives of the histidine residue which can be used to functionalize the glutamate units are identical to or different from one another and can be e.g. histidine esters (such as the methyl ester and the ethyl ester), histidinol and histamine. These derivatives can also be e.g. histidinamide, the N-monomethyl derivative of histidinamide and the N,N′-dimethyl derivative of histidinamide.

In one variant of the invention, at least one of the hydrophobic groups [GH4] is included in a hydrophobic graft comprising at least one spacer R¹² for joining the hydrophobic group [GH4] to a polyglutamate chain (e.g. a poly-glutamate main chain or skeleton). This spacer can comprise e.g. at least one direct covalent bond and/or at least one amide linkage and/or at least one ester linkage. For example, the spacer can be of the type belonging to the group comprising, in particular, different amino acid residues from the constituent monomeric unit of the polyglutamate, amino alcohol derivatives, polyamine (e.g. diamine) derivatives, polyol (e.g. diol) derivatives and hydroxy acid derivatives.

The spacers R¹² forming hydrophobic grafts with the hydrophobic groups [GH4] can be di-, tri- or tetravalent (or even pentavalent or higher). In the case of a divalent spacer R¹², the hydrophobic graft contains a single group [GH4], whereas a trivalent spacer R¹² gives the hydrophobic graft a bifid character, i.e. the hydrophobic graft comprises two hydrophobic groups [GH4]. Examples of a trivalent spacer R¹² which may be mentioned, inter alia, are an amino acid residue, e.g. glutamic acid, or a polyol residue, e.g. glycerol. Thus two advantageous but non-limiting examples of hydrophobic grafts comprising hydrophobic groups [GH4] are dialkyl glycerols and dialkyl glutamates.

For further details on the preparation and synthesis of polyamino acids of formula (V), reference may usefully be made to patent application FR 2 892 725.

In one variant of the invention, the hydrophobic groups [GH1], [GH2] and [GH3] of the amphiphilic polyamino acid of general formula (I), (II), (III) or (IV) are selected from the group comprising octyloxy, dodecyloxy, tetradecyloxy, hexadecyloxy, octadecyloxy, oleyloxy, tocopheryloxy and cholesteryloxy radicals. Furthermore, the hydrophobic groups [GH4] of the amphiphilic polyamino acid of general formula (V) are selected from the group comprising octyl, dodecyl, tetradecyl, hexadecyl, octadecyl, oleyl, tocopheryl and cholesteryl radicals. Preferably, in this variant of the invention, R⁴ (formula I), R⁷ (formulae II and IV) and R¹¹ (formulae III and IV) are a direct bond and R¹² (formula V) is a group —O—.

In another variant of the invention, optionally combined with the previous variant, the hydrophobic groups [GH1], [GH2], [GH3] and [GH4] of the amphiphilic polyamino acid (I), (II), (III), (IV) or (V) independently of one another can also each be a monovalent group of general formula (VI) below:

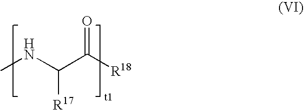

in which:

R¹⁷ independently of one another are a methyl, isopropyl, isobutyl, sec-butyl or benzyl group;

R¹⁸ independently of one another are a hydrophobic group containing from 6 to 30 carbon atoms; and

t1 varies from 0 to 6.

In one variant of the invention, all or some of the hydrophobic groups R¹⁸ are selected independently of one another from:

a linear or branched alkoxy group containing from 6 to 30 carbon atoms and optionally containing at least one unit of unsaturation and/or at least one heteroatom,

an alkoxy group containing from 6 to 30 carbon atoms, having one or more fused carbocycles and optionally containing at least one unit of unsaturation and/or at least one heteroatom, and

an alkoxyaryl group having 7 to 30 carbon atoms or an aryloxyalkyl group containing from 7 to 30 carbon atoms and optionally containing at least one unit of unsaturation and/or at least one heteroatom.

In general, the heteroatoms that can be found are oxygen, nitrogen or sulfur atoms. In practice and without implying a limitation, in the amphiphilic polyamino acid of general formula (I), (II), (III), (IV) or (V), the group R¹⁸ of the hydrophobic group of formula (VI) is selected from the group comprising octyloxy, dodecyloxy, tetradecyloxy, hexadecyloxy, octadecyloxy, oleyloxy, tocopheryloxy and cholesteryloxy radicals.

In one variant of the invention, the molar grafting rate of hydrophobic groups [GH1], [GH2], [GH3] and [GH4] is between 1 mol % and 25 mol %. Preferably, the molar grafting rate is between 5 mol % and 20 mol %. The molar grafting rate of hydrophobic groups is defined as the ratio of the number of amino acid residues in the main chain which carry at least one hydrophobic group, to the total number of amino acid residues in the main chain of the amphiphilic polyamino acid (the degree of polymerization).

In one variant of the invention, the amphiphilic polyamino acid also carries at least one graft of the polyalkylene glycol type bonded to a glutamate and/or aspartate residue. Advantageously, this graft has general formula (VII) below:

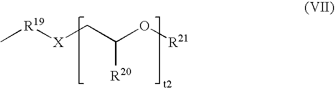

in which:

R¹⁹ independently of one another are a direct bond or a spacer group comprising from 1 to 4 amino acid residues;

X is a heteroatom selected from the group comprising oxygen, nitrogen and sulfur;

R²⁰ and R²¹ independently of one another are a hydrogen atom or a linear C1 to C4 alkyl group; and

t2 varies from 10 to 1000 and preferably from 50 to 300.

In practice, the polyalkylene glycol is e.g. a polyethylene glycol. It is desirable for the molar grafting percentage of polyalkylene glycol to vary from 1 to 30 mol %.

In practice, the pharmaceutically acceptable salts of the amphiphilic polymer used according to the invention, especially the amphiphilic polyamino acids of general formula (I), (II), (III), (IV) or (V), are those in which the carboxylic acid groups are in ionized form in aqueous solution. Salification is generally effected with a metal or organic cation such as:

the following metal cations: sodium, potassium, calcium or magnesium ions;

the following organic cations: cations based on amine, cations based on oligo-amine and cations based on polyamine, especially polyethyleneimine. Among cations based on amino acid(s), one preferred variant consists in selecting cations based on lysine or arginine, such as polylysine or oligolysine.

The amphiphilic polyamino acids described above are valuable because, with an adjustable grafting rate, they disperse in water at pH 7.4 (e.g. with a phosphate buffer) to give colloidal suspensions.

Furthermore, active principles such as proteins, peptides or small molecules, examples of which are given below, can associate spontaneously with these polyamino acids. This association can result from various non-covalent physico-chemical interactions. These can be e.g. hydrogen bonds, ionic bonds, hydrophobic interactions, Van der Waals forces or several of these non-covalent bonds simultaneously. In some cases it is possible that, strictly speaking, there is no association between the active principles and the polyamino acids, but simply a steric trapping of the active principle in a physical gel of polyamino acid.

The amphiphilic polyamino acids that are capable of being used in the formulation of the invention are obtained e.g. by methods known to those skilled in the art. Random polyamino acids can be obtained by grafting the hydrophobic group [GH], previously functionalized with the “spacer”, directly onto the polymer by a conventional coupling reaction. Block or multiblock polyamino acids can be obtained by sequential polymerization of the corresponding amino acid N-carboxy anhydrides (NCA).

For example, a homopolyglutamate or homopolyaspartate polyamino acid or a block, multiblock or random glutamate/aspartate copolymer is prepared by conventional methods.

To obtain a polyamino acid of the alpha type, the most common technique is based on the polymerization of amino acid N-carboxy anhydrides (NCA), which is described e.g. in the article entitled “Biopolymers” by Fuller, W. D., Verlander, M. S, and Goodman, M.: “A procedure for the facile synthesis of amino acid N-carboxy anhydrides”, 1976, 15, 1869, and in the book by H. R. Kricheldorf entitled “Alpha-amino acid N-carboxy anhydride and related heterocycles”, Springer Verlag (1987). The NCA derivatives are preferably NCA-O-Me, NCA-O-Et or NCA-O-Bz derivatives (Me=methyl, Et=ethyl and Bz=benzyl). The polymers obtained are then hydrolyzed under appropriate conditions to give the polymer in its acid form. These methods are based on the description given in patent FR-A-2 801 226 to the Applicant. A number of polymers that can be used according to the invention, e.g. of the poly(alpha-L-aspartic), poly(alpha-L-glutamic), poly(alpha-D-glutamic) and poly(gamma-L-glutamic) types of variable molecular weights, are commercially available. The polyaspartic of the alpha-beta type is obtained by the condensation of aspartic acid (to give a polysuccinimide), followed by basic hydrolysis (cf. Tomida et al., Polymer, 1997, 38, 4733-36).

Coupling of the hydrophobic graft [GH] with an acid group of the polymer is easily effected by reacting the polyamino acid in the presence of a carbodiimide as coupling agent, and optionally a catalyst such as 4-dimethylaminopyridine, in an appropriate solvent such as dimethylformamide (DMF), N-methylpyrrolidone (NMP) or dimethyl sulfoxide (DMSO). The carbodiimide is e.g. dicyclohexylcarbodiimide or diisopropylcarbodiimide. The grafting rate is controlled chemically by the stoichiometry of the constituents and reactants or by the reaction time. The hydrophobic grafts [GH] functionalized with a “spacer” are obtained by conventional peptide coupling or by direct condensation under acid catalysis. These techniques are well known to those skilled in the art.

A block or multiblock copolymer is synthesized using NCA derivatives previously synthesized with the hydrophobic graft. For example, the hydrophobic NCA derivative is copolymerized with NCA-O-benzyl and the benzyl groups are then selectively removed by hydrolysis.

### Continuous Lipid Phase

For the purposes of the invention, a lipid or oily phase comprises a hydrophobic organic compound that is liquid at a temperature between 20° C. and 40° C., or a mixture of such compounds. Preferably, the lipid phase comprises at least one oil selected from metabolizable oils. Furthermore, the dynamic viscosity at 25° C. of the chosen oil or mixture of oils is less than or equal to 400 mPa·s. It can be assumed that the injectability of the pharmaceutical composition will improve as the dynamic viscosity of the lipid phase decreases. It is therefore preferable to use a lipid phase whose dynamic viscosity at 25° C. is less than or equal to 150 mPa·s; even better, in increasing order of preference, it is less than or equal to 80 mPa·s, 40 mPa·s or 30 mPa·s.

Among the useful metabolizable oils, there may be mentioned oils selected from triglycerides of a medium-chain fatty acid of animal, vegetable or synthetic origin, fatty acids of animal or vegetable origin, their esters and their salts, and mixtures thereof. For example, glycerol tricaprylate/caprate (e.g. Miglyol® 812, Sasol®) is used as an oil of the triglyceride of a medium-chain fatty acid. As another example, the lipid phase can comprise at least one oil selected from olive oil, sweet-almond oil, sunflower oil, soybean oil, groundnut oil, maize oil, coconut oil, cottonseed oil, castor oil and mixtures thereof.

### Surfactant

The surfactant or mixture of surfactants is selected so as to have an HLB below 6.

The hydrophilic-lipophilic balance (HLB) is an empirical measure of the degree of hydrophilicity or lipophilicity of a molecule. Various methods of measuring HLB have been described, especially by Griffin (1949): “Classification of Surface-Active Agents by ‘HLB’”, Journal of the Society of Cosmetic Chemists 1: 311, Griffin (1954): “Calculation of HLB Values of Non-Ionic Surfactants”, Journal of the Society of Cosmetic Chemists 5: 259, or Davies (1957): “A quantitative kinetic theory of emulsion type, I. Physical chemistry of the emulsifying agent”, Gas/Liquid and Liquid/Liquid Interface. Proceedings of the International Congress of Surface Activity, 426-438.

For example, the surfactant is selected from the group comprising polyglyceryl esters, ricinoleic acid esters, sorbitan oleate, lecithin, mono- and diglycerides of C6 to C12 fatty acids and/or unsaturated fatty acids, polyricinoleic acid esters and mixtures thereof.

Preferably, the surfactant comprises polyglyceryl esters, especially those of natural fatty acids such as oleic, stearic, ricinoleic, linoleic and linolenic acids. Polyglyceryl ricinoleates, or even polyglyceryl polyricinoleates (PGPR), are preferred surfactants.

### Preparation of the Pharmaceutical Composition

In a first step, an aqueous phase is prepared which contains at least one amphiphilic polymer at a concentration of between 5 and 100 mg per g of aqueous phase, and at least one active principle. For example, in the case of interferon alpha-2b as the active principle, provision can be made for a concentration of 1 mg per g of aqueous phase. The aqueous phase is stirred at 25° C. for a sufficient time to allow the amphiphilic polymer and the active principle to associate, e.g. 24 h. A lipid phase is then prepared by solubilizing a surfactant or mixture of surfactants whose HLB is below 6 in a metabolizable oil or mixture of metabolizable oils whose viscosity at 25° C. is less than or equal to 400 mPa·s. Preferably, the viscosity at 25° C. of the oil or mixture of oils is below 100 mPa·s.

The aqueous phase and the lipid phase are brought into contact for approximately 1 h, with moderate stirring, ensuring that the weight ratio of aqueous phase to lipid phase is less than or equal to 50/50 and preferably less than or equal to 30/70. The aqueous phase is dispersed in the lipid phase by means of a rotor-stator homogenizer or a high-pressure homogenizer.

Preferably, the pharmaceutical composition contains an excess of at least 10% by weight of lipid phase, based on the amount of lipid phase required to cause inversion of the emulsion at 25° C. An example of a method of measuring the inversion point of an emulsion is the conductimetric method described in particular in the following reference: Puisieux F., Seiller M. (1983). “Galenica 5: Les Systemes Dispersés—Agents de Surface et Émulsions (Disperse Systems—Surface-active Agents and Emulsions)”, vol. 1. Paris: Technique et Documentation—Lavoisier. With a disperse aqueous phase, a continuous lipid phase and a surfactant at a given temperature, there is weight ratio between the disperse aqueous phase and the continuous lipid phase at which the emulsion inverts, i.e. changes from an emulsion of the water-in-oil type to an emulsion of the oil-in-water type. This ratio is called the inversion point of the emulsion.

An excess of lipid phase, based on the amount of lipid phase below which the water-in-oil emulsion inverts, allows to prevent this inversion under storage conditions. This excess also makes it possible to limit the viscosity of the water-in-oil emulsion.

Thus it is particularly preferable to use an excess of at least 15% by weight of lipid phase, based on the amount of lipid phase required to cause inversion of the emulsion at 25° C.; even better, in increasing order of preference, the excess of lipid phase is at least 20% by weight or even 30% by weight.

In the same connection, the weight ratio of aqueous disperse phase to continuous lipid phase is less than or equal to 50:50 in the pharmaceutical composition, preferably less than or equal to 40:60 and particularly preferably less than or equal to 30:70. In fact, the greater the proportion of continuous lipid phase, the more the viscosity of the water-in-oil emulsion depends on the viscosity of the continuous lipid phase.

Preferably, the pharmaceutical composition has a dynamic viscosity at 25° C. which is less than or equal to 200 mPa·s. Even better, in increasing order of preference, the dynamic viscosity at 25° C. of the pharmaceutical composition is less than or equal to 150 mPa·s or less than or equal to 100 mPa·s.

The aqueous phase contains at least one amphiphilic polymer and at least one active principle. The dynamic viscosity at 25° C. of the aqueous phase before dispersion in the continuous lipid phase can be greater than or equal to 20 mPa·s. The aqueous phase can also take the form of a physical gel dispersed in the continuous lipid phase.

The pharmaceutical composition can be injected by the parenteral route. The injectability test is described in the Examples.

### Active Principle

In general, the active principle(s) is (are) selected from proteins, glycoproteins, proteins bonded to one or more polyalkylene glycol chains (e.g. PEGylated proteins, i.e. proteins bonded to one or more polyethylene glycol chains), peptides, polysaccharides, liposaccharides, steroids, oligonucleotides, polynucleotides and mixtures thereof.

In one preferred variant of the invention, at least one active principle is hydrophilic.

More precisely, the active principle AP can be selected from the group comprising erythropoietin, ocytocin, vasopressin, adrenocorticotropic hormone, epidermal growth factor, platelet-derived growth factor (PDGF), hemopoiesis stimulating factors, factors VIII and IX, hemoglobin, cytochromes, albumins, prolactin, luliberin or gonadotropin releasing hormone (LHRH), LHRH antagonists, LHRH agonists, human, porcine or bovine growth hormones (GH), growth hormone releasing factor, insulin, somatostatin, glucagon, interleukins (IL) such as IL-2, IL-11, IL-12 and mixtures thereof, α-, β- or γ-interferon (IFN) and mixtures thereof, gastrin, tetragastrin, pentagastrin, urogastrone, secretin, calcitonin, enkephalins, endomorphines, angiotensins, thyrotropin releasing hormone (TRH), tumor necrosis factors (TNF), nerve growth factor (NGF), granulocyte colony stimulating factor (G-CSF), granulocyte macrophage colony stimulating factor (GM-CSF), macrophage colony stimulating factor (M-CSF), heparinase, bone morphogenic proteins (BMP), human atrial natriuretic peptide (hANP), glucagon-like peptide (GLP-1), vascular endothelial growth factor (VEGF), recombinant hepatitis B surface antigen (rHBsAg), renin, cytokines, bradykinin, bacitracins, polymixins, colistins, tyrocidine, gramicidines, cyclosporins and synthetic analogs, pharmaceutically active modifications and fragments of enzymes, cytokines and antibodies, and mixtures thereof.

In one variant, the active principle is a small hydrophobic, hydrophilic or amphiphilic organic molecule of the type belonging to the anthracycline, taxoid or camptothecin families or of the type belonging to the peptide family, such as leuprolide or cyclosporin, and mixtures thereof. For the purposes of the present disclosure, a small molecule is especially a small non-protein molecule, e.g. a small molecule devoid of amino acids.

In another variant, the active principle can be selected from at least one of the following families of active substances: agents for treating alcohol abuse, agents for treating Alzheimer's disease, anesthetics, agents for treating acromegaly, analgesics, antiasthmatics, agents for treating allergies, anticancer agents, anti-inflammatories, anticoagulants and antithrombotics, anticonvulsants, antiepileptics, antidiabetics, antiemetics, antiglaucomas, antihistamines, anti-infectives, antibiotics, antifungals, antivirals, antiparkinsonians, anticholinergics, antitussives, carbonic anhydrase inhibitors, cardiovascular agents, hypolipemics, antiarrhythmics, vaso-dilators, antianginals, antihypertensives, vasoprotectors, cholinesterase inhibitors, agents for treating central nervous system disorders, central nervous system stimulants, contraceptives, fertility promoters, labor inducers and inhibitors, agents for treating cystic fibrosis, dopamine receptor agonists, agents for treating endo-metriosis, agents for treating erectile dysfunctions, agents for treating fertility, agents for treating gastrointestinal disorders, immunomodulators and immuno-suppressants, agents for treating memory disorders, antimigraines, muscle relaxants, nucleoside analogs, agents for treating osteoporosis, parasympathomimetics, prostaglandins, psychotherapeutic agents, sedatives, hypnotics and tranquilizers, neuroleptics, anxiolytics, psychostimulants, antidepressants, agents for treating dermatological disorders, steroids and hormones, amphetamines, anorexics, non-analgesic painkillers, antiepileptics, barbiturates, benzodiazepines, hypnotics, laxatives, psychotropics and any associations of these products.

The invention further relates to a method of therapeutic treatment that consists essentially in administering the composition as described in the present disclosure by the oral, nasal, ocular, cutaneous, vaginal, rectal or parenteral route. Among the parenteral routes, there may be mentioned subcutaneous injection, intramuscular injection, intraperitoneal injection, intradermal injection, intravenous injection, intra-arterial injection, intraspinal injection, intra-articular injection and intrapleural injection.

### BRIEF DESCRIPTION OF THE FIGURES

FIG. 1: Viscosity of the solution of amphiphilic polyamino acid (black triangles) and the suspension of amphiphilic polyamino acid in oil (black squares) of Example 3.

FIG. 2: Injectability measurement on a suspension of amphiphilic polyamino acid with 25 G needles (black squares) and 27 G needles (black triangles) of Example 3.

FIG. 3: In vitro release of methylene blue from a suspension without amphiphilic polyamino acid (black triangles) and with amphiphilic polyamino acid (black squares) of Example 4.

FIG. 4: Variation in viscosity at 10 s⁻¹ of the suspension as a function of time (Example 6).

FIG. 5: Variation in diameter (d50%) of the aqueous droplets as a function of time (Example 6).

### EXAMPLES

### Example 1

### Preparation of the Suspension

An amphiphilic polyamino acid is prepared as follows: The alpha-L-polyglutamate polymer, having a molecular weight equivalent to about 10,000, relative to a polyoxyethylene standard, is obtained by the polymerization of NCAGluOMe, followed by hydrolysis, as described in patent application FR 2 801 226. 5.5 g of this alpha-L-polyglutamate polymer are solubilized in 92 ml of dimethylformamide (DMF) by heating at 40° C. for 2 hours. Once the polymer has been solubilized, the temperature is allowed to fall to 25° C. and 1.49 g of D-alpha-tocopherol of natural origin (98.5%, obtained from ADM France), previously solubilized in 6 ml of DMF, 0.09 g of 4-dimethylaminopyridine, previously solubilized in 6 ml of DMF, and 0.57 g of diisopropylcarbodiimide, previously solubilized in 6 ml of DMF, are added in succession. After stirring for 8 hours at 25° C., the reaction medium is poured into 800 ml of water containing 15% of sodium chloride and hydrochloric acid (pH 2). The polymer which precipitates is then recovered by filtration and washed with 0.1 N hydrochloric acid and then with water. The polymer is subsequently resolubilized in 75 ml of DMF and then reprecipitated in water containing salt and acid at pH 2, as above. After 2 washes with water, the polymer is washed several times with diisopropyl ether. It is then dried in a vacuum oven at 40° C. to give a yield in the order of 85%.

The grafting rate estimated by proton NMR is about 5.2% and HPLC analysis reveals a residual tocopherol content of less than 0.3%. The weight-average molecular weight Mw, measured by GPC with NMP as the eluent, is 17,500 g/mol (in polymethyl methacrylate equivalents).

15 ml of an aqueous solution of the amphiphilic polyamino acid synthesized above, containing 22 mg/g, and 35 ml of a solution of glycerol tricaprylate/caprate (Miglyol 812, Sasol), comprising 5% (by weight) of polyglyceryl polyricinoleate (Grindsted™ PGPR 90, Danisco), are prepared separately. These two phases are subsequently brought into contact and then emulsified by means of an Ultra-Turrax T8 rotor-stator homogenizer, Ika-Werke, for 1 minute with an S8N-5G probe at a speed of 20,000 min⁻¹, or by means of a high-pressure homogenizer (Emulsiflex C3, Avestin), after 3 passes of 1000 bar to give a suspension of aqueous droplets of amphiphilic polyamino acid in a continuous phase of glycerol tricaprylate/caprate having a weight ratio of aqueous phase to lipid phase of 30/70.

### Example 2

### Stability of the Active Principle Improved by the Presence of the Polyamino Acid

A suspension is prepared according to the protocol described in Example 1, a protein, namely interferon alpha-2b (IFNα2b), being incorporated into the aqueous phase at a concentration of 1 mg/g.

The IFNα2b in the suspension is quantified by a sandwich ELISA (IM3193 kit, Beckman Coulter). After the suspension containing the IFNα2b and the amphiphilic polyamino acid has been kept for one week at 37° C., the ELISA, after extraction, gives an 85% recovery of the IFNα2b, compared with the same emulsion kept for one week at 5° C. Under the same storage conditions, but in the absence of amphiphilic polyamino acid, the recovery is only 6%.

### Example 3

### Variation in Viscosity/Injectability

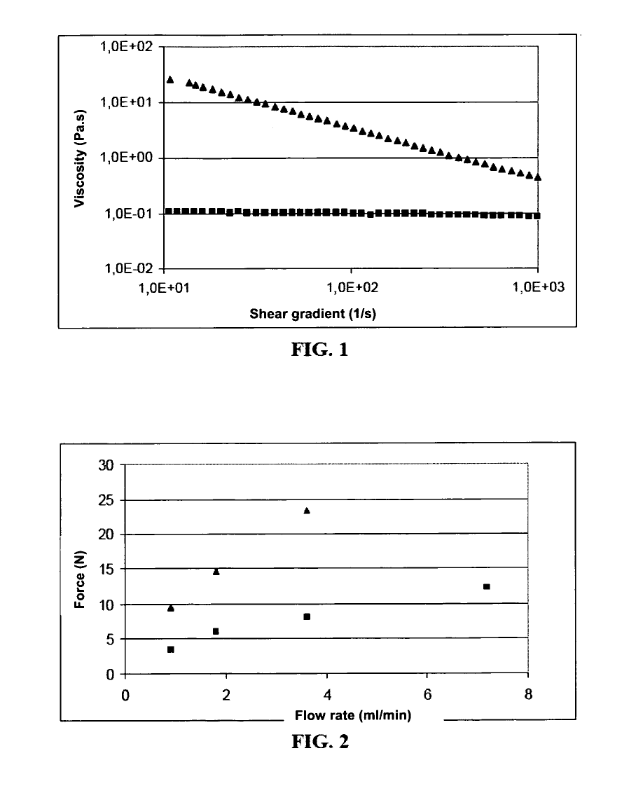

An aqueous phase of amphiphilic polyamino acid at a concentration of 50 mg/ml (which is a physical gel at this concentration) is dispersed in the lipid phase according to the procedure described in Example 1 to give a suspension of hydrogel. The viscosity value was measured by comparison for the suspension and the initial gel. This measurement is made by characterizing the change in viscosity as a function of shear gradient (from 10 to 1000 s⁻¹) at 25° C. using a stress-controlled rheometer (Gemini, Bohlin) with cone-and-plate geometry installed (2 cm or 4 cm and 1° angle). The comparison shows that, at low shear gradients (10 s⁻¹), the suspension is characterized by a viscosity in the order of 0.1 Pa·s, which is about 250 times lower than the viscosity of the initial gel (cf. FIG. 1).

The injectable character of this suspension was evaluated by the injectability test IT. This test consists in measuring the force that has to be applied to the piston of a syringe in order to obtain a given flow rate at the syringe outlet. An injectable formulation is understood as meaning a formulation which, after evaluation by this injectability test IT, is characterized by a force below 25 N for a flow rate of 3.5 ml/min.

The suspension described in Example 3 is introduced into a 1 ml syringe (Injekt-F, Braun) to which a 25 G or 27 G needle has been fitted. This syringe is placed in a traction apparatus (DY34, Adamel Lhomargy). The injectability test is performed.

As seen from the results shown in FIG. 2, it is found that the suspension of amphiphilic polyamino acid in a continuous lipid phase is injectable with 25 G and 27 G needles according to this test, which is not the case for the initial hydrogel.

### Example 4

### Prolonged Release Behavior

The aim is to determine in vitro the proportion of active principle released into the physiological medium by a suspension according to the invention, as a function of time, and to compare it with that observed in the absence of amphiphilic polyamino acid.

A first suspension is prepared according to the protocol described in Example 1, the water-soluble dye methylene blue (Sigma) being incorporated at a concentration of 0.01% (by weight) in the aqueous phase.

The dye simulates the active principle for these in vitro experiments. A second suspension is prepared in the absence of amphiphilic polyamino acid, methylene blue again being incorporated at 0.01% (by weight). 50 μl of each of these two suspensions are injected into 4 ml of 0.1 M phosphate buffer solution (PBS: Phosphate Buffer Saline, Sigma), which simulates the physiological medium. Each preparation is stirred at a temperature of 37° C. Sixty microliters of continuous phase are withdrawn at different times and then replaced with 60 μl of 0.1 M PBS. The concentration of methylene blue in the different aliquots is measured by UV-visible spectroscopy at 550 nm (Lambda 35 UV/Vis Spectrometer, Perkin-Elmer Instruments). It is thus possible to determine the proportion of dye released into the continuous phase as a function of time. Under these conditions the observed behavior shows that the release of the dye is delayed: whereas about 70% of the methylene blue is released in 14 days in the case of the suspension not comprising amphiphilic polyamino acid, the presence of amphiphilic polyamino acid in the aqueous drops makes it possible to reduce the release rate, since the proportion of dye released is only in the order of 20% after 14 days under the same conditions.

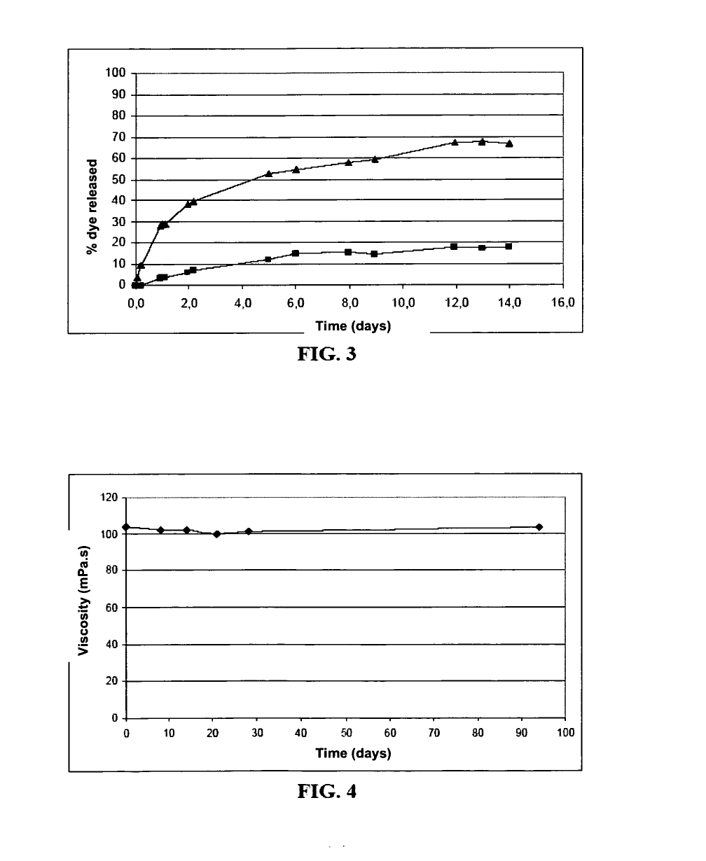

These results are shown in FIG. 3.

### Example 5

An additional experiment consisted in preparing a suspension according to the protocol of Example 1, a therapeutic protein, namely human growth hormone (hGH: human Growth Hormone, Prospec), being incorporated at a concentration of 5 mg/g into the aqueous phase before dispersion in the lipid phase. Fifty microliters of product are injected into 4 ml of 0.1 M phosphate buffer (PBS: Phosphate Buffer Saline, Sigma). Sixty microliters of continuous phase are withdrawn at different times and then replaced with 60 μl of 0.1 M PBS. The whole preparation is stirred at a temperature of 37° C.

The concentration of hGH in the external aqueous phase is measured by liquid chromatography (HPLC, C18 column).

The results obtained showed that, after 5 days at 37° C., the proportion of protein released into the external aqueous phase is less than 2%.

### Example 6

### Physical Stability

An aqueous phase of amphiphilic polyamino acid at a concentration of 20 mg/ml is dispersed in the lipid phase according to the procedure described in Example 1. The resulting suspension is kept at 5° C. and characterized as a function of time by measuring the viscosity and the size of the aqueous droplets.

The viscosity is measured by determining the viscosity value for a shear gradient of 10 s⁻¹ at 25° C. using a stress-controlled rheometer (Gemini, Bohlin) with cone-and-plate geometry installed (2 cm or 4 cm and 1° angle).

The size is measured by laser diffraction with a granulometer (Mastersizer 200, Malvern), using heptane (SDS) as the dispersion medium.

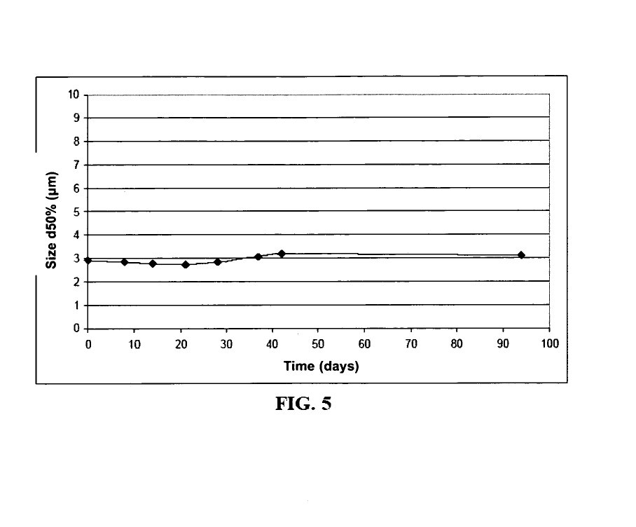

The results (FIGS. 4 and 5) show the stability of these suspensions, since there is no significant variation in the viscosity or the size of the aqueous droplets after more than 3 months at 5° C.

1. Pharmaceutical composition for the prolonged release of at least one active principle, comprising at least one active principle in an aqueous phase containing at least one amphiphilic polymer, said aqueous phase being in the form of a dispersion in a continuous lipid phase, the composition being in the form of a water-in-oil emulsion comprising:
a pharmaceutically acceptable, continuous lipid phase,
an aqueous disperse phase containing at least one amphiphilic polymer and at least one active principle not covalently bonded to said amphiphilic polymer, and
at least one pharmaceutically acceptable surfactant.

2. Pharmaceutical composition according to claim 1 wherein said amphiphilic polymer is an amphiphilic polymer carrying at least one hydrophobic group.

3. Pharmaceutical composition according to claim 1 or 2 wherein said amphiphilic polymer is an amphiphilic polyamino acid.

4. Pharmaceutical composition according to claim 3 wherein said amphiphilic polyamino acid carries at least one hydrophobic group.

5. Pharmaceutical composition according to claim 1 or 2 wherein said amphiphilic polymer is a polysaccharide carrying at least one hydrophobic group.

6. Pharmaceutical composition according to any one of the preceding claims, characterized in that it can be administered by a route selected from the following routes: oral, nasal, ocular, cutaneous, vaginal, rectal and parenteral.

7. Pharmaceutical composition according to any one of the preceding claims wherein at least one active principle is selected from proteins, glycoproteins, proteins bonded to one or more polyalkylene glycol chains, peptides, poly-saccharides, liposaccharides, steroids, oligonucleotides, polynucleotides and mixtures thereof.

8. Pharmaceutical composition according to claim 7 wherein at least one active principle is hydrophilic.

9. Pharmaceutical composition according to claim 7 wherein at least one active principle is selected from erythropoietin, ocytocin, vasopressin, adrenocorticotropic hormone, epidermal growth factor, platelet-derived growth factor (PDGF), hemopoiesis stimulating factors, factor VIII, factor IX, hemoglobin, cytochromes, albumins, prolactin, luliberin or gonadotropin releasing hormone (LHRH), LHRH antagonists, LHRH agonists, human growth hormones (GH), porcine GH, bovine GH, somatoliberin, insulin, somatostatin, glucagon, interleukins (IL-2, IL-11, IL-12), α-, β- or γ-interferon, gastrin, tetragastrin, pentagastrin, urogastrone, secretin, calcitonin, enkephalins, endomorphines, angiotensins, thyrotropin releasing hormone (TRH), tumor necrosis factors (TNF), nerve growth factor (NGF), granulocyte colony stimulating factor (G-CSF), granulocyte macrophage colony stimulating factor (GM-CSF), macrophage colony stimulating factor (M-CSF), heparinase, bone morphogenic proteins (BMP), human atrial natriuretic peptide (hANP), glucagon-like peptide (GLP-1), vascular endothelial growth factor (VEGF), renin, cytokines, bradykinin, bacitracins, polymixins, colistins, tyrocidine, gramicidines, cyclosporins and synthetic analogs, pharmaceutically active modifications and fragments of enzymes, cytokines and antibodies, and mixtures thereof.

10. Pharmaceutical composition according to claim 3 or 4 wherein said amphiphilic polyamino acid is a polymer, a sequence or random copolymer or a mixture of such polymers and/or copolymers.

11. Pharmaceutical composition according to claim 10 wherein the main chain of the amphiphilic polyamino acid contains monomers derived from glutamic acid and/or aspartic acid, at least some of the monomers carrying at least one pendant hydrophobic group.

12. Pharmaceutical composition according to claim 10 or 11 wherein the main chain of the amphiphilic polyamino acid contains monomers derived from glutamic acid and/or aspartic acid, at least some of the monomers carrying a pendant group derived from a histidine residue.

13. Pharmaceutical composition according to claim 12 wherein at least one pendant group derived from a histidine residue is bonded to a glutamic residue via an amide linkage.

14. Pharmaceutical composition according to claim 12 or 13 wherein the pendant groups derived from a histidine residue are identical to or different from one another and are selected from histidine, histidine esters, histidinol, histamine, histidinamide, N-methylhistidinamide and N,N′-dimethylhistidinamide.

15. Pharmaceutical composition according to claim 10 or 11 wherein said amphiphilic polyamino acid (PAA) has general formula (I) below, and its pharmaceutically acceptable salts: 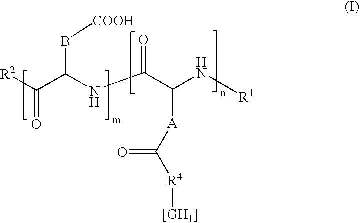
in which:
R¹ is a hydrogen atom, a linear C2 to C10 acyl group, a branched C3 to C10 acyl group, a pyroglutamate group or a group —R⁴-[GH1];
R² is a group —NHR⁵ or a terminal amino acid residue bonded via the nitrogen, whose acid group(s) is (are) optionally modified by an amine —NHR⁵ or an alcohol —OR⁶;
R⁴ independently of one another are a direct bond or a spacer group comprising from 1 to 4 amino acid residues;
R⁵ is a hydrogen atom, a linear C1 to C10 alkyl group, a branched C3 to C10 alkyl group or a benzyl group;
R⁶ is a hydrogen atom, a linear C1 to C10 alkyl group, a branched C3 to C10 alkyl group, a benzyl group or a group —R⁴-[GH1];
A and B independently of one another are a group —CH₂— (aspartic residue) or —CH₂—CH₂— (glutamic residue);
[GH1] is a hydrophobic group;
the molar grafting rate of hydrophobic groups [GH1], n/(n+m), is sufficiently low for the amphiphilic polyamino acid to form a colloidal suspension of submicronic particles of polyamino acid when it is in solution in water at pH 7 and at 25° C., n/(n+m) being from 1 to 25 mol %; and
the degree of polymerization (n+m) varies from 10 to 1000.

16. Pharmaceutical composition according to claim 10 or 11 wherein said amphiphilic polyamino acid (PAA) has one of general formulae (II), (III) and (IV) below, and their pharmaceutically acceptable salts: 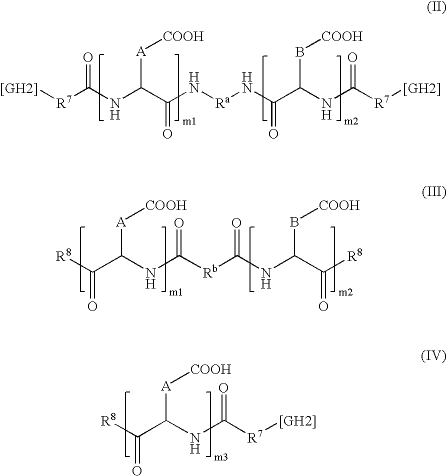
in which:
R^{a} is a linear C2 to C6 alkylene group;
R^{b} is a C2 to C6 alkylene group, a C2 to C6 dialkoxy group or a C2 to C6 diamine group;
R⁷ independently of one another are a direct bond, a spacer group comprising from 1 to 4 amino acid residues, or a group —C(O)—CH₂—CH₂—;
R⁸ are a group —NHR⁹ or a terminal amino acid residue bonded via the nitrogen, whose acid group(s) is (are) optionally modified by an amine —NHR⁹ or an alcohol —OR¹⁰, respectively;
R⁹ is a hydrogen atom, a linear C1 to C10 alkyl group, a branched C3 to C10 alkyl group or a benzyl group;
R¹⁰ is a hydrogen atom, a linear C1 to C10 alkyl group, a branched C3 to C10 alkyl group, a benzyl group or a group —R¹¹-[GH3];
R¹¹ independently of one another are a direct bond or a spacer group comprising from 1 to 4 amino acid residues;
A and B independently of one another are a group —CH₂— (aspartic residue) or —CH₂—CH₂— (glutamic residue);
[GH2] and [GH3] independently of one another are a hydrophobic group; and
the degrees of polymerization (m1+m2) and m3 vary from 10 to 1000.

17. Pharmaceutical composition according to any one of claims 10 to 14 wherein said amphiphilic polyamino acid (PAA) has general formula (V) below, and its pharmaceutically acceptable salts: 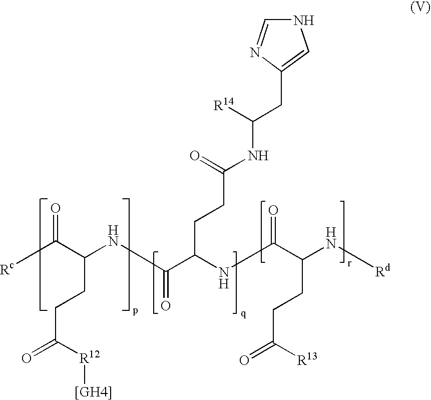
in which:
R^{c} is a group —NHR¹⁵ or a terminal amino acid residue bonded via the nitrogen, whose acid group(s) is (are) optionally modified by an amine —NHR⁵ or an alcohol —OR¹⁶, respectively;
R^{d} is a hydrogen atom, a linear C2 to C10 acyl group, a branched C3 to C10 acyl group or a pyroglutamate group;
R¹² independently of one another are a divalent, trivalent or tetravalent linking group selected from the following groups: —O—, —NH—, C1 to C5-N-alkyl, an amino acid residue, a C2 to C6 diol, a C3 to C6 triol, a C2 to C6 diamine, a C3 to C6 triamine, a C2 to C6 amino alcohol or a C2 to C6 hydroxy acid;
R¹³ independently of one another are a group —OH or an ethanolamine group bonded via the amine fraction;
R¹⁴ is an alkyl ester group, a group —CH₂OH (histidinol), a hydrogen atom (histamine), a group —C(O)NH₂ (histidinamide), a group —C(O)NHCH₃ or a group —C(O)N(CH₃)₂;
R¹⁵ and R¹⁶ independently of one another are a hydrogen atom, a linear C1 to C10 alkyl group, a branched C3 to C10 alkyl group or a benzyl group;
[GH4] independently of one another are each a hydrophobic group selected from:
linear or branched C8 to C30 alkyl groups optionally containing at least one unit of unsaturation and/or at least one heteroatom,
C8 to C30 alkylaryl or arylalkyl groups optionally containing at least one unit of unsaturation and/or at least one heteroatom, and
C8 to C30 (poly)cyclic groups optionally containing at least one unit of unsaturation and/or at least one heteroatom;
p, q and r are positive integers;
the molar grafting rate of hydrophobic groups [GH], (p)/(p+q+r), varies from 1 to 50 mol %, with the proviso that each copolymer chain has an average of at least 3 hydrophobic grafts;
the molar grafting rate of groups derived from the histidine residue, (q)/(p+q+r), varies from 1 to 99 mol %;
(r)/(p+q+r) varies from 0 to 98 mol %; and
(p+q+r) varies from 10 to 1000.

18. Pharmaceutical composition according to claim 15, 16 or 17 wherein said hydrophobic group [GH1], [GH2] and [GH3] are selected from the group comprising octyloxy, dodecyloxy, tetradecyloxy, hexadecyloxy, octadecyloxy, oleyloxy, tocopheryloxy and cholesteryloxy radicals, wherein said hydrophobic groups [GH4] are selected from the group comprising octyl, dodecyl, tetradecyl, hexadecyl, octadecyl, oleyl, tocopheryl and cholesteryl radicals, and wherein R⁴, R⁷ and R¹¹ are a direct bond and R¹² is a group —O—.

19. Pharmaceutical composition according to claim 15, 16 or 17 wherein the hydrophobic groups [GH1], [GH2], [GH3] and [GH4] independently of one another are each a monovalent group of general formula (VI) below: 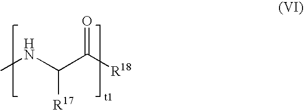
in which:
R¹⁷ independently of one another are a methyl, isopropyl, isobutyl, sec-butyl or benzyl group;
R¹⁸ independently of one another are a hydrophobic group containing from 6 to 30 carbon atoms; and
t1 varies from 0 to 6.

20. Pharmaceutical composition according to claim 19 wherein the hydrophobic groups R¹⁸ are selected independently of one another from:
a linear or branched alkoxy group containing from 6 to 30 carbon atoms and optionally containing at least one unit of unsaturation and/or at least one heteroatom,
an alkoxy group containing from 6 to 30 carbon atoms, having one or more fused carbocycles and optionally containing at least one unit of unsaturation and/or at least one heteroatom, and
an alkoxyaryl group having 7 to 30 carbon atoms or an aryloxyalkyl group containing from 7 to 30 carbon atoms and optionally containing at least one unit of unsaturation and/or at least one heteroatom.

21. Pharmaceutical composition according to claim 19 or 20 wherein said hydrophobic group R¹⁸ is selected from the group comprising octyloxy, dodecyloxy, tetradecyloxy, hexadecyloxy, octadecyloxy, oleyloxy, tocopheryloxy and cholesteryloxy radicals.

22. Pharmaceutical composition according to any one of claims 10 to 21 wherein the main chain of the amphiphilic polyamino acid contains monomers derived from glutamic acid and/or aspartic acid, said polyamino acid carrying at least one graft of the polyalkylene glycol type.

23. Pharmaceutical composition according to claim 22 wherein the graft of the polyalkylene glycol type has general formula (VII) below: 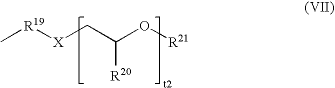
in which:
R¹⁹ independently of one another are a direct bond or a spacer group comprising from 1 to 4 amino acid residues;
X is a heteroatom selected from the group comprising oxygen, nitrogen and sulfur;
R²⁰ and R²¹ independently of one another are a hydrogen atom or a linear C1 to C4 alkyl group; and
t2 varies from 10 to 1000.

24. Pharmaceutical composition according to claim 22 or 23 wherein the polyalkylene glycol is a polyethylene glycol.

25. Pharmaceutical composition according to claim 22, 23 or 24 wherein the amphiphilic polyamino acid has a molar grafting rate of polyalkylene glycol varying from 1 to 30 mol %.

26. Pharmaceutical composition according to any one of claims 10 to 17 wherein the main chain of the amphiphilic polyamino acid (PAA) is an alpha-L-glutamate or alpha-L-glutamic homopolymer.

27. Pharmaceutical composition according to any one of claims 10 to 16 wherein the main chain of the amphiphilic polyamino acid (PAA) is an alpha-L-aspartate or alpha-L-aspartic homopolymer.

28. Pharmaceutical composition according to any one of claims 10 to 16 wherein the main chain of the amphiphilic polyamino acid (PAA) is an alpha-L-aspartate/alpha-L-glutamate or alpha-L-aspartic/alpha-L-glutamic copolymer.

29. Pharmaceutical composition according to claim 15 or 17 wherein the molar grafting rate of hydrophobic groups is between 1 mol % and 25 mol %.

30. Pharmaceutical composition according to any one of claims 10 to 29 wherein the molecular weight of the amphiphilic polyamino acid (PAA) is between 2000 and 100,000 g/mol.

31. Pharmaceutical composition according to any one of the preceding claims wherein the surfactant or mixture of surfactants has an HLB below 6.

32. Pharmaceutical composition according to claim 31 wherein the surfactant is selected from the group comprising polyglyceryl esters, ricinoleic acid esters, sorbitan oleate, lecithin, mono- and diglycerides of C6 to C12 fatty acids and/or unsaturated fatty acids, polyricinoleic acid esters, polyglyceryl polyricinoleate and mixtures thereof.

33. Pharmaceutical composition according to any one of the preceding claims wherein the lipid phase comprises at least one oil selected from metabolizable oils, the dynamic viscosity at 25° C. of said oil or mixture of oils being less than or equal to 400 mPa·s.

34. Pharmaceutical composition according to claim 33 wherein the lipid phase comprises at least one oil selected from triglycerides of a medium-chain fatty acid of animal, vegetable or synthetic origin, fatty acids of animal or vegetable origin, their esters and their salts, and mixtures thereof.

35. Pharmaceutical composition according to claim 33 or 34 wherein the lipid phase comprises at least one oil selected from olive oil, sweet-almond oil, sunflower oil, soybean oil, groundnut oil, maize oil, coconut oil, cottonseed oil, castor oil and mixtures thereof.

36. Pharmaceutical composition according to any one of the preceding claims containing an excess of at least 10% by weight of lipid phase, based on the amount of lipid phase required to cause inversion of the emulsion at 25° C., measured by the conductimetric method.

37. Pharmaceutical composition according to any one of the preceding claims wherein the weight ratio of aqueous disperse phase to continuous lipid phase is less than or equal to 50:50.

38. Pharmaceutical composition according to any one of the preceding claims having a dynamic viscosity at 25° C. which is less than or equal to 200 mPa·s, the aqueous phase being in the form of a physical gel or having a dynamic viscosity at 25° C. which is greater than or equal to 20 mPa·s.

39. Pharmaceutical composition according to any one of the preceding claims containing from 5 to 100 mg of amphiphilic polyamino acid per gram of aqueous phase.
